(19) United States
(12) Patent Application Publication
(10) Pub. No.: US 2025/0002973 A1
(43) Pub. Date: Jan. 2, 2025

(54) METHODS AND FORMULATIONS FOR DENATURING DNA

(71) Applicant: Illumina, Inc., San Diego, CA (US)

(72) Inventors: Dale Weekes, Haverhill (GB); Oliver Miller, Linton (GB)

(73) Assignee: Illumina, Inc., San Diego, CA (US)

(21) Appl. No.: 18/758,012

(22) Filed: Jun. 28, 2024

Related U.S. Application Data
(60) Provisional application No. 63/511,076, filed on Jun. 29, 2023.

(51) Int. Cl. C12Q 1/6806

(52) U.S. Cl. CPC C12Q 1/6806

(57) ABSTRACT

Disclosed herein are formulations and methods for denaturing DNA that utilize betaine as the denaturant. In some examples, the formulations include additives other than betaine, such as, for example, dimethyl sulfoxide (DMSO) and/or diethylene glycol (DEG).

### CROSS-REFERENCE TO RELATED APPLICATIONS

This application claims the benefit of U.S. Provisional Patent Application No. 63/511,076, filed Jun. 29, 2023 and entitled “Methods and Formulations for Denaturing DNA,” the entire contents of which are incorporated by reference herein.

### FIELD

This application relates to methods and formulations for denaturing DNA.

### BACKGROUND

Formamide and sodium hydroxide are currently used in a number of sequencing systems to both denature the DNA library before sequencing and de-hybridise sequencing strands during clustering/resynthesis between reads However, neither formamide nor sodium hydroxide are ideal denaturing agents. Formamide is a hazardous substance. It is a suspected carcinogen and may cause birth defects. It, therefore, necessitates specialized disposal routes, hinders efforts to reach Corporate Social Responsibility goals, and is a key complaint from customers. Also, manual denaturation by NaOH increases the hands-on time of sequencing, is a potential source of human error, and requires the customer to purchase and store additional chemicals and equipment. Operator error during denaturation is a frequent issue for customers using sodium hydroxide and is a significant cause of customer run failures. Additionally, sodium hydroxide solution is hazardous to users because of its corrosive properties and cannot be used in cartridges without additional measures such as alkaline-resistant foils.

### SUMMARY

Some examples herein provide a solution formulation for denaturing double-stranded DNA (dsDNA), the solution formulation including betaine; and at least one organic solvent.

In some examples, the at least one organic solvent includes diethylene glycol (DEG).

In some examples, a concentration of betaine in the solution formulation is between about 1M and about 5M, and a concentration of DEG in the solution formulation is between about 3% and about 40%.

In some examples, the concentration of DEG in the solution formulation is between about 20% and about 40%. In some examples, the concentration of DEG in the solution formulation is between about 20% and about 30%. In some examples, the concentration of DEG in the solution formulation is about 20%.

In some examples, the concentration of betaine in the solution formulation is between about 2M and about 5M. In some examples, the concentration of betaine in the solution formulation is between about 2M and about 4M. In some examples, the concentration of betaine in the solution formulation is about 2M. In some examples, the concentration of betaine in the solution formulation is about 3M.

In some examples, the at least one organic solvent includes diethylene glycol (DEG) and dimethyl sulfoxide (DMSO).

In some examples, a concentration of betaine in the solution formulation is between about 1M and about 5M, and a concentration of the DEG in the solution formulation is between about 3% and about 40%.

In some examples, a concentration of DMSO in the solution formulation is between about 5% and about 30%. In some examples, the concentration of the DMSO in the solution formulation is between about 7% and about 12%. In some examples, the concentration of DMSO in the solution formulation is about 10%. In some examples, the concentration of DMSO in the solution formulation is between about 17% and about 22%. In some examples, the concentration of DMSO in the solution formulation is about 20%.

In some examples, the concentration of the DEG in the solution formulation is between about 5% and about 25%. In some examples, the concentration of the DEG in the solution formulation is between about 13% and about 17%. In some examples, the concentration of the DEG in the solution formulation is about 15%.

In some examples, the concentration of betaine in the solution formulation is between about 2M and about 3M. In some examples, the concentration of betaine in the solution formulation is about 2M. In some examples, the concentration of betaine in the solution formulation is about 2.5M. In some examples, the concentration of betaine in the solution formulation is about 3M.

In some examples, the at least one organic solvent includes DMSO.

In some examples, a concentration of betaine in the solution formulation is between about 1M and about 5M. In some examples, the concentration of betaine in the solution formulation is between about 2M and about 3M. In some examples, the concentration of betaine in the solution formulation is about 2M. In some examples, the concentration of betaine in the solution formulation is about 2.5M. In some examples, the concentration of betaine in the solution formulation is about 3M.

In some examples, a concentration of DMSO in the solution formulation is between about 5% and about 30%. In some examples, the concentration of the DMSO in the solution formulation is between about 7% and about 12%. In some examples, the concentration of DMSO in the solution formulation is about 10%. In some examples, the concentration of DMSO in the solution formulation is between about 17% and about 22%. In some examples, the concentration of DMSO in the solution formulation is about 20%.

In some examples, any of the solution formulations further include a zwitterionic buffer. In some examples, the zwitterionic buffer produces a pH in the solution formulation at a range of between about 9.5 and about 11.5. In some examples, the zwitterionic buffer includes any one or more of 4. (cyclohexylamino)-1-butanesulfonic acid (CABS), N-cyclohexyl-2-aminoethanesulfonic acid (CHES), 3-(cyclohexylamino)-2-hydroxy-1-propanesulfonic acid (CAPSO), N-cyclohexyl-3-aminopropanesulfonic acid (CAPS), and 3-((3-cholamidopropyl)dimethylammonio)-1-propanesulfonate) (CHAPS).

Some examples herein provide a method of denaturing double-stranded DNA (dsDNA), the method including: contacting the dsDNA in a fluid-filled container with a denaturant including betaine.

In some examples, a ratio of denaturant to the dsDNA is between about 1:1 and about 3:1. In some examples, a ratio of denaturant to the dsDNA is about 1:1. In some examples, the ratio of the denaturant to the dsDNA is about 1.3:1. In some examples, the ratio of the denaturant to the dsDNA is about 1.5:1. In some examples, the ratio of the denaturant to the dsDNA is about 2:1. In some examples, the ratio of the denaturant to the dsDNA is about 2.5:1. In some examples, the ratio of the denaturant to the dsDNA is about 3:1.

In some examples, the denaturant further includes diethylene glycol (DEG).

In some examples, the denaturant further includes dimethyl sulfoxide (DMSO).

In some examples, the denaturant further includes a zwitterionic buffer. In some examples, the zwitterionic buffer produces a pH in the fluid-filled container between about 9.5 and about 11.5.

Some examples herein provide a kit for denaturing double-stranded DNA (dsDNA), the kit including: a denaturant including betaine and diethylene glycol (DEG) in a solution; and a buffer.

In some examples, a concentration of DEG in the solution is between about 20% and about 40%.

In some examples, the denaturant further includes dimethyl sulfoxide (DMSO).

In some examples, the concentration of DEG in the solution is between about 5% and about 20%.

In some examples, a concentration of betaine in the solution is between about 1M and 5M. In some examples, the concentration of betaine in the solution is between about 2M and about 3.5M. In some examples, the concentration of betaine in the solution is about 2M. In some examples, the concentration of betaine in the solution is about 2.5M. In some examples, the concentration of betaine in the solution is about 3.0M. In some examples, the concentration of betaine in the solution is about 3.5M.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF THE DRAWINGS

FIGS. 1A-1C show comparable sequencing metrics for denaturant formulations containing betaine v. a formamide control (LDR).

FIG. 2 shows data from a standard 2×151 sequencing run using DNA denaturant containing betaine that is DMSO-free.

FIG. 3 shows that primary metrics were not significantly different when TruSeq DNA Nano human libraries that were denatured with formulations containing betaine v. standard formamide (LDR).

FIG. 4 shows metrics for DNA denaturant formulations containing betaine that are DMSO-free compared to formulations that contain DMSO.

FIGS. 5A-5C show sequencing data obtained using a DNA denaturant formulation that contains betaine and diethylene glycol (DEG) compared to using the denaturant formamide (LDR).

FIGS. 6A and 6B show sequencing data in which DNA denaturant formulations that contained only betaine were compared to DNA denaturant formulations that contained additional components other than betaine. FIG. 6A shows data of denaturing after clustering. FIG. 6B shows data of denaturing DNA libraries.

FIGS. 7A and 7B show data of denaturing DNA libraries using various DNA denaturant formulations that contain various concentrations of DEG and DMSO, as well as other additives.

FIG. 8 shows data of denaturing DNA after clustering using various DNA denaturant formulations containing different concentrations of buffer with different pH ranges.

FIG. 9 shows data of denaturing DNA after clustering using various DNA denaturant formulations with different concentrations of betaine.

FIG. 10 shows data of denaturing DNA using various XDR formulations using a surface hybridization-based assay.

FIG. 11 shows data of GC coverage using various XDR formulations to denature DNA during a sequencing reaction.

FIG. 12 shows data of global coverage when various XDR formulations when various concentrations of betaine and DMSO were used to denature DNA during sequencing reactions.

FIG. 13 shows sequencing data when using various XDR formulations to denature DNA in a HiSeq X sequencing run.

FIG. 14 shows data of sequencing intensity of various sequencing reads using the XDR14 formulation as the denaturant.

FIG. 15 shows data of various sequencing metrics using various XDR formulations in an HiSeq X sequencing run.

FIG. 16 shows data of various sequencing metrics using various XDR formulations with and without DEG, in an HiSeq X sequencing run.

FIG. 17 shows data of various sequencing metrics using various XDR formulations with and without CAPS buffer, in an HiSeq X sequencing run.

FIG. 18 shows data of the viscosity of XDR formulations compared to LDR.

FIG. 19 shows data of the viscosity of various XDR formulations and the viscosity of most of the high-performance XDR formulations (see insert).

### DETAILED DESCRIPTION

Current DNA denaturants include formamide and sodium hydroxide. Formamide is a serious health hazard and sodium hydroxide requires manual denaturation that can cause human error, in addition to being corrosive. Disclosed herein are alternative denaturing reagents (XDR formulations) that can be used to denature DNA. XDR formulations disclosed herein are not hazardous and can be used to effectively denature template DNA before sequencing, and/or to de-hybridize sequencing primers that are bound to surface DNA during sequencing.

In some examples, XDR formulations disclosed herein include betaine as well as other additive(s). In some examples, the XDR denaturants may include any one or more of a buffer, diethylene glycol (DEG), and dimethyl sulfoxide (DMSO). In some examples, the XDR denaturants include betaine and DEG. In some examples, the XDR denaturants include betaine, DEG, and a buffer. In some examples, the XDR formulations include betaine and DMSO. In some examples, the XDR formulations include betaine, DMSO, and a buffer. In some examples, the XDR formulations including betaine, DEG, and DMSO. In some examples, the XDR formulations include betaine, DEG, DMSO, and a buffer.

In some examples, any of the XDR formulations described herein can be used to denature libraries before sequencing. In some examples, any of the XDR formulations described herein can be used to de-hybridise strands in clustering and/or resynthesis, between reads. In some examples, any of the XDR formulations described herein can be used in bridge amplification. In some examples, any of the XDR formulations described herein can be used in exclusion amplification.

First, some terms used herein will be briefly explained. Then, some example compositions and example methods using the compositions will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components

The terms “substantially,” “approximately,” and “about” used throughout this specification are used to describe and account for small fluctuations, such as due to variations in processing. For example, they may refer to less than or equal to ±10% of the stated amount, such as less than or equal to ±5% of the stated amount, such as less than or equal to ±2% of the stated amount, such as less than or equal to ±1% of the stated amount, such as less than or equal to +0.5% of the stated amount, such as less than or equal to ±0.2% of the stated amount, such as less than or equal to ±0.1% of the stated amount, such as less than or equal to ±0.05% of the stated amount.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include inosine, xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 5-halouracil, 5-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “XDR”, the phrase “XDR solution formulation”, or the phrase “XDR formulation”, each refer to a DNA denaturant formulation in which betaine is used as the primary additive to denature the DNA. In some examples, an XDR formulation includes additional component(s) such as any one or more of a buffer, di-ethylene glycol (DEG), and dimethyl sulfoxide (DMSO). Various “XDR formulations” are disclosed herein; these various “XDR formulations” are labeled, for example, as XDR1, XDR2, XDR3, etc. . . .

As used herein, the term “betaine” is intended to refer to glycine betaine, also known as trimethylglycine (TMG), and has the following structure:

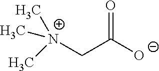

When a composition or solution herein is referred to as including a range % or a particular % of a compound, it is meant that such compound is present at that range % or particular % by volume.

As used herein, the term “LDR” refers to a denaturant that includes 99.9% formamide.

### XDR Solution Formulations for Denaturing Double-Stranded DNA

Some examples herein provide a solution formulation for denaturing double-stranded DNA (dsDNA) that includes betaine. In some examples, the solution formulation de-hybridizes a primer bound to surface DNA.

In some examples, the solution formulation includes betaine. In some examples, the solution formulation includes betaine and diethylene glycol (DEG). In some examples, the solution formulation is an XDR formulation.

In some examples, a concentration of betaine in the solution formulation is between about 1M and about 5M, for example about 1M, about 1.1M, about 1.2M, about 1.3M, about 1.4M, about 1.5M, about 1.6M, about 1.7M, about 1.8M, about 1.9M, about 2M, about 2.1M, about 2.2M, about 2.3M, about 2.4M, about 2.5M, about 2.6M, about 2.7M, about 2.9M, about 3.0M, about 3.1M, about 3.2M, about 3.3M, about 3.4M, about 3.5M, about 3.6M, about 3.7M, about 3.8M, about 3.9M, about 4.0M, about 4.1M, about 4.2M, about 4.3M, about 4.4M, about 4.5M, about 4.6M, about 4.7M, about 4.8M, about 4.9M, or about 5M. In some examples, the concentration of betaine in the solution formulation is between about 2M and about 5M. In some examples, the concentration of betaine in the solution formulation is between about 2M and about 4M. In some examples, the concentration of betaine in the solution formulation is between about 2M and about 3.5M. In some examples, the concentration of betaine in the solution formulation is less than 1M. In some examples, the concentration of betaine in the solution formulation is greater than 5M.

In some examples, a concentration of DEG in the solution formulation is between about 3% and about 40%. In some examples, the concentration of DEG in the solution formulation is between about 20% and about 40%, for example about 20%, about 21%, about 22%, about 23%, about 24%, about 25%, about 26%, about 27%, about 28%, about, 29%, about 30%, about 31%, about 32%, about 33%, about 34%, about 35%, about 36%, about 37%, about 38%, about 39%, or about 40%. In some examples, the concentration of DEG in the solution formulation is greater than 40%. In some examples, the concentration of DEG in the solution formulation is between about 20% and about 30%.

In some examples, the solution formulation further includes dimethyl sulfoxide (DMSO). In some examples, a concentration of DMSO in the solution formulation is between about 5% and about 30%, for example, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%. In some examples, the concentration of the DMSO in the solution formulation is between about 7% and about 12%. In some examples, the concentration of DMSO in the solution formulation is about 7%, about 8%, about 9%, about 10%, about 11%, about 12%, about 13%, about 14%, or about 15%. In some examples, the concentration of DMSO in the solution formulation is between about 17% and about 22%. In some examples, the concentration of DMSO in the solution formulation is about 17%, about 18%, about 19%, about 20%, about 21%, or about 22%.

In some examples, a concentration of the DEG is between about 5% and about 25% in any of the solution formulations containing DMSO described herein. In some examples, the concentration of DEG is about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25%. In some examples, the concentration of the DEG in the solution formulation is between about 13% and about 17%. In some examples, the concentration of the DEG in the solution formulation is about 13%, about 14%, about 15%, about 16%, or about 17%.

In some examples, a concentration of betaine is between about 2M and 3M in any of the solution formulations containing DMSO described herein. In some examples, the concentration of betaine in the solution formulation is about 2M, about 2.1M, about 2.2M, about 2.3M, about 2.4M, about 2.5M, about 2.6M, about 2.7M, about 2.8M, about 2.9M, or about 3M.

In some examples, any of the solution formulations described herein further includes a buffer. In some examples, the buffer includes a zwitterionic buffer. In some examples, the zwitterionic buffer produces a pH in the solution formulation at a range of between about 9.5 and about 11.5. In some examples, the zwitterionic buffer produces a pH in the solution formulation at a range of between about 8.5 and about 12.5. In some examples, the zwitterionic buffer produces a pH in the solution formulation of about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10, about 10.1, about 10.2, about 10.3, about 10.4, about 10.5, about 10.6, about 10.7, about 10.8, about 10.9, about 11, about 11.1, about 11.2, about 11.3, about 11.4, about 11.5, about 11.6, about 11.7, about 11.8, about 11.9, about 12.0, about 12.1, about 12.2, about 12.3, about 12.4, or about 12.5.

In some examples, the zwitterionic buffer includes any one or more of 4-(cyclohexylamino)-1-butanesulfonic acid (CABS), N-cyclohexyl-2-aminoethanesulfonic acid (CHES), 3-(cyclohexylamino)-2-hydroxy-1-propanesulfonic acid (CAPSO), N-cyclohexyl-3-aminopropanesulfonic acid (CAPS), and 3-((3-cholamidopropyl)dimethylammonio)-1-propanesulfonate) (CHAPS). In some examples, the zwitterionic buffer includes any zwitterionic buffer known in the art.

In some examples, the solution formulations described herein are any of XDR1, XDR2, XDR3, XDR4, XDR5, XDR6, XDR7, XDR8, XDR9, XDR10, XDR11, XDR12, XDR13, XDR14, XDR15, XDR16, XDR17, XDR18, XDR19, XDR20, XDR21, XDR22, XDR23, XDR24, XDR25, XDR26, XDR27, and XDR30. The components that make up each of these XDR formulations, which are aqueous solutions, are as follows:

### XDR1

3M betaine
20% DMSO
15% DEG

### XDR2

3M betaine
15% DMSO
20% DEG

### XDR3

3M betaine
15% DMSO
15% DEG

### XDR4

2.5M betaine
20% DMSO
15% DEG

### XDR5

2.5M betaine
15% DMSO
20% DEG

### XDR6

2.5M betaine
15% DMSO
15% DEG

### XDR7

3M betaine
20% DMSO
15% DEG

### XDR8

3M betaine
10% DMSO
15% DEG

### XDR9

3M betaine
15% DEG

### XDR10

2M betaine
20% DMSO
15% DEG

### XDR11

2M betaine
10% DMSO
15% DEG

### XDR12

2M betaine
15% DMSO

### XDR13

2.5M betaine
10% DMSO

### XDR14

2.5M betaine
15% DMSO
5% DEG

### XDR15

2.5M betaine
20% DMSO
5% DEG

### XDR16

3M betaine
20% DMSO

### XDR17

2.5M betaine
15% DMSO
10% DEG

### XDR18

2.5M betaine
20% DMSO

### XDR19

3M betaine
15% DMSO
5% DEG

### XDR20

50 mM CAPS pH 10.5
3M betaine
20% DMSO
5% DEG

### XDR21

50 mM CAPS pH 10.5
3.5M betaine
20% DMSO

### XDR22

50 mM CAPS pH 10.5
3M betaine
20% DMSO
10% DEG

### XDR23

50 mM CAPS pH 11
2.5M betaine
10% DMSO
20% DEG

### XDR24

50 mM CAPS pH 11
3M betaine
20% DMSO

### XDR25

50 mM CAPS pH 11
3M betaine
20% DMSO
5% DEG

### XDR26

50 mM CAPS pH 11
4M betaine
20% DMSO

### XDR27

4M betaine
20% DMSO

### XDR30

50 mM Tris pH 9
4M betaine
20% DMSO

### Methods of Denaturing Double-Stranded DNA

Some examples herein provide a method of denaturing double-stranded DNA (dsDNA), including contacting the dsDNA in a fluid-filled container with a denaturant that includes betaine. In some examples, the denaturant that includes betaine is any of the XDR formulations described herein.

In some examples, a ratio of the denaturant to the dsDNA is between about 1:1 and about 3:1. Illustratively, in some examples, a ratio of the denaturant to the dsDNA is about 1:1. In some examples, the ratio of denaturant to the dsDNA is about 1.2:1. In some examples, the ratio of the denaturant to the dsDNA is about 1.3:1. In some examples, the ratio of the denaturant to the dsDNA is about 1.4:1. In some examples, the ratio of the denaturant to the dsDNA is about 1.5:1. In some examples, the ratio of the denaturant to the dsDNA is about 1.6:1. In some examples, the ratio of the denaturant to the dsDNA is about 2:1. In some examples, the ratio of the denaturant to the dsDNA is about 2.5:1. In some examples, the ratio of the denaturant to the dsDNA is about 3:1.

In some examples, the denaturant further includes diethylene glycol (DEG). In some examples, the concentration of DEG is between about 3% and about 40%, for example, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%. In some examples, the concentration of DEG in the solution is less than 3%. In some examples, the concentration of DEG in the solution is greater than 40%.

In some examples, the denaturant further includes dimethyl sulfoxide (DMSO). In some examples, a concentration of DMSO in the solution formulation is between about 5% and about 30%, for example, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%.

In some examples, the denaturant further includes a zwitterionic buffer. In some examples, the zwitterionic buffer produces a pH in the fluid-filled container between about 9.5 and about 11.5. In some examples, the zwitterionic buffer produces a pH in the solution formulation at a range of between about 8.5 and about 12.5. In some examples, the zwitterionic buffer produces a pH in the solution formulation of about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10, about 10.1, about 10.2, about 10.3, about 10.4, about 10.5, about 10.6, about 10.7, about 10.8, about 10.9, about 11, about 11.1, about 11.2, about 11.3, about 11.4, about 11.5, about 11.6, about 11.7, about 11.8, about 11.9, about 12.0, about 12.1, about 12.2, about 12.3, about 12.4, or about 12.5.

In some examples, the zwitterionic buffer includes any one or more of 4-(cyclohexylamino). 1-butanesulfonic acid (CABS), N-cyclohexyl-2-aminoethanesulfonic acid (CHES), 3-(cyclohexylamino)-2-hydroxy-1-propanesulfonic acid (CAPSO), N-cyclohexyl-3-aminopropanesulfonic acid (CAPS), and 3-((3-cholamidopropyl)dimethylammonio)-1-propanesulfonate) (CHAPS). In some examples, the zwitterionic buffer includes any zwitterionic buffer known in the art.

Some examples herein provide a kit for denaturing double-stranded DNA (dsDNA), including a denaturant that includes betaine and diethylene glycol (DEG) in a solution; and a buffer.

In some examples, a concentration of DEG in the solution is between about 20% and about 40%. In some examples, the concentration of DEG in the solution is about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%.

In some examples, the denaturant further includes dimethyl sulfoxide (DMSO). In some examples, the concentration of DEG in the solution is between about 5% and about 20%. In some examples, the concentration of DEG in the solution is about 5%, about 6%, about 7%, about 8%, about 9%, about 10%, about 11%, about 12%, about 13%, about 14%, about 15%, about 16%, about 17%, about 18%, about 19%, or about 20%.

In some examples, any of the kits described herein include a concentration of betaine in the solution that is between about 1 M and 5 M. In some examples, the concentration of betaine in the solution is about 2 M, about 2.5 M, about 3.0 M, about 3.5 M, about 4.5 M, or about 5.0 M.

In some examples, the buffer includes a zwitterionic buffer. In some examples, the zwitterionic buffer produces a pH in the solution formulation at a range of between about 9.5 and about 11.5. In some examples, the zwitterionic buffer produces a pH in the solution formulation at a range of between about 8.5 and about 12.5. In some examples, the zwitterionic buffer produces a pH in the solution formulation of about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10, about 10.1, about 10.2, about 10.3, about 10.4, about 10.5, about 10.6, about 10.7, about 10.8, about 10.9, about 11, about 11.1, about 11.2, about 11.3, about 11.4, about 11.5, about 11.6, about 11.7, about 11.8, about 11.9, about 12.0, about 12.1, about 12.2, about 12.3, about 12.4, or about 12.5.

In some examples, the zwitterionic buffer includes any one or more of 4-(cyclohexylamino)-1-butanesulfonic acid (CABS), N-cyclohexyl-2-aminoethanesulfonic acid (CHES), 3-(cyclohexylamino)-2-hydroxy-1-propanesulfonic acid (CAPSO), N-cyclohexyl-3-aminopropanesulfonic acid (CAPS), and 3-((3-cholamidopropyl)dimethylammonio)-1-propanesulfonate) (CHAPS). In some examples, the zwitterionic buffer includes any zwitterionic buffer known in the art.

### Amplification Methods

The XDR solution formulations, the solution formulations, and the methods of denaturing double-stranded DNA described herein can be used in the context of amplification methods, such as bridge amplification and exclusion amplification. Bridge amplification and exclusion amplification can be carried out in a flow cell.

Bridge Amplification: Bridge amplification can be performed on a flow cell. Single stranded template DNA is hybridised to lawn primers in a flow cell, and a polymerase is used to extend the primer to form double-stranded DNA. The double-stranded DNA is denatured, and the original template strand of the DNA molecule is washed away. This results in a single-stranded DNA molecule being bound to the lawn primers of the flow cell. The single-stranded DNA molecule turns over and forms a “bridge” by hybridising to a nearby lawn primer that is complementary to a sequence of the single-stranded DNA molecule. Polymerase extends the hybridised primer resulting in bridge amplification of the DNA molecule and the creation of a double-stranded DNA molecule. The double-stranded DNA molecule is then denatured.

The double-stranded DNA molecule can be denatured using (i) any of the XDR solution formulations described herein, (ii) any of the solution formulations described herein, or (iii) any of the methods of denaturing double-stranded DNA described herein. Denaturing results in two copies of single-stranded templates, one of which is immobilised to the support and the other of which may be washed away.

The one strand that is immobilised on the support may be used in further bridge amplification operations so as to generate a cluster that subsequently may be sequenced. Cluster generation produces clusters of double-stranded DNA. These clusters can be denatured using (i) any of the XDR solution formulations described herein, (ii) any of the solution formulations described herein, or (iii) any of the methods of denaturing double-stranded DNA described herein.

Exclusion Amplification: Exclusion amplification methods may allow for the amplification of a single target polynucleotide per substrate region and the production of a substantially monoclonal population of amplicons in a substrate region. For example, the rate of amplification of the first captured target polynucleotide within a substrate region may be more rapid relative to much slower rates of transport and capture of target polynucleotides at the substrate region. As such, the first target polynucleotide captured in a substrate region may be amplified rapidly and fill the entire substrate region, thus inhibiting the capture of additional target polynucleotide in the same substrate region. Alternatively, if a second target polynucleotide attaches to same substrate region after the first polynucleotide, the relatively rapid amplification of the first polynucleotide may fill enough of the substrate region to result in a signal that is sufficiently strong to perform sequencing by synthesis (e.g., the substrate region may be at least functionally monoclonal). The use of exclusion amplification may also result in super-Poisson distributions of monoclonal substrate regions; that is, the fraction of substrate regions in an array that are functionally monoclonal may exceed the fraction predicted by the Poisson distribution.

Exclusion amplification can generate clusters of double-stranded DNA. The double-strand DNA can be denatured using (i) any of the XDR solution formulations described herein, (ii) any of the solution formulations described herein, or (iii) any of the methods of denaturing double-stranded DNA described herein.

### Sequencing of Template Strands

In some examples, sequencing occurs after cluster generation of DNA. In some examples, the DNA clusters are produced from bridge amplification or from exclusion amplification. In some examples, the DNA clusters are attached to the surface of a flow cell. To facilitate sequencing, it is preferable if one of the strands of DNA is removed from the surface to allow efficient hybridization of a sequencing primer to the remaining immobilised strand. Any of the solution formulations, XDR solution formulations, or any of the methods of denaturing double-stranded DNA described herein can be used to de-hybridized DNA, thereby allowing removal of a strand of DNA from the surface.

Sequence data can be obtained from both ends of a template duplex by obtaining a sequence read from one strand of the template from a primer in solution, copying the strand using immobilised primers, releasing the first strand and sequencing the second, copied strand. For example, sequence data can be obtained from both ends of the immobilised duplex by a method wherein the duplex is treated to free a 3′-hydroxyl moiety that can be used an extension primer. The extension primer can then be used to read the first sequence from one strand of the template. After the first read, the strand can be extended to fully copy all the bases up to the end of the first strand. This second copy remains attached to the surface at the 5′-end. If the first strand is removed from the surface, the sequence of the second strand can be read. This gives a sequence read from both ends of the original fragment. The process whereby the strand is regenerated after the first read is known as “Paired-end resynthesis” or “PE resynthesis”. The typical steps of pairwise sequencing are known and have been described in WO 2008/041002, the entire contents of which are incorporated by reference herein.

Sequencing can be carried out using any suitable “sequencing-by-synthesis” technique, wherein nucleotides are added successively to the free 3′ hydroxyl group, resulting in synthesis of a polynucleotide chain in the 5′ to 3′ direction. The nature of the nucleotide added is preferably determined after each addition. One particular sequencing method relies on the use of modified nucleotides that can act as reversible chain terminators. Such reversible chain terminators include removable 3′ blocking groups. Once such a modified nucleotide has been incorporated into the growing polynucleotide chain complementary to the region of the template being sequenced there is no free 3′-OH group available to direct further sequence extension and therefore the polymerase cannot add further nucleotides. Once the nature of the base incorporated into the growing chain has been determined, the 3′ block may be removed to allow addition of the next successive nucleotide. By ordering the products derived using these modified nucleotides it is possible to deduce the DNA sequence of the DNA template. Such reactions can be done in a single experiment if each of the modified nucleotides has attached thereto a different label, known to correspond to the particular base, to facilitate discrimination between the bases added at each incorporation step. Suitable labels are described in PCT application PCT/GB2007/001770, the entire contents of which are incorporated by reference herein. Alternatively, a separate reaction may be carried out containing each of the modified nucleotides added individually.

Any of the solution formulations, XDR solution formulations, or any of the methods of denaturing double-stranded DNA described herein can be used to remove sequencing primers from strands of DNA.

### Working Examples

The examples provided below are include for illustrative purposes and are not meant to be limiting of any of the disclosures described herein.

### Example 1—Methods of Testing Denaturants in Sequencing Reactions

New denaturant solutions with various XDR formulations described in Examples 2-6 below were tested during sequencing reactions. The libraries were sequenced at 2×151 cycles on a modified HiSeq X instrument and then tested on a NextSeq 2000. The sequencing was performed with standard workflows and reagents, except with new denaturant solutions (XDR formulations) replacing LDR in the cartridge, reagent plate, or reagent tubes.

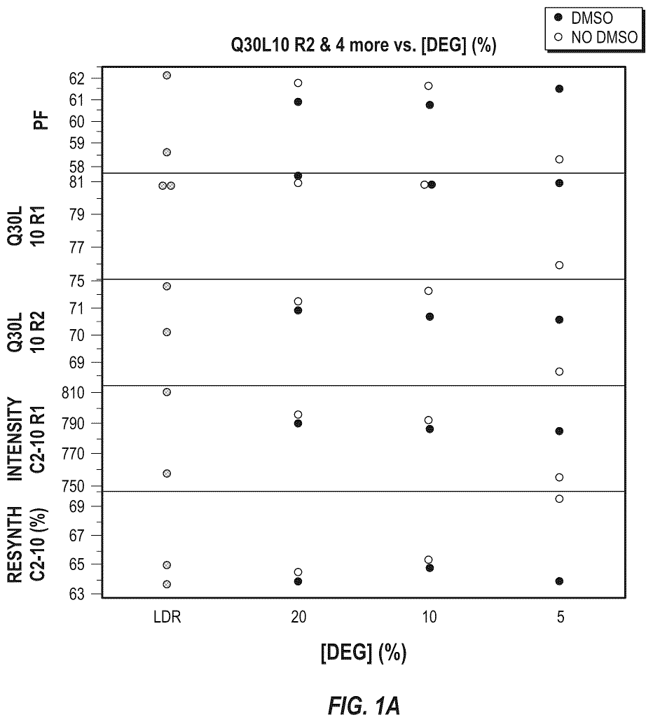

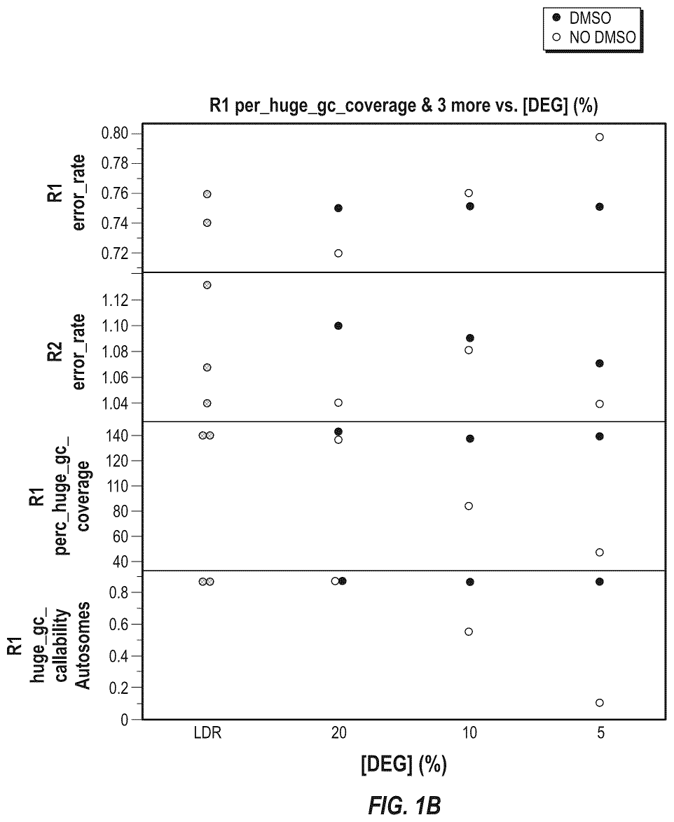

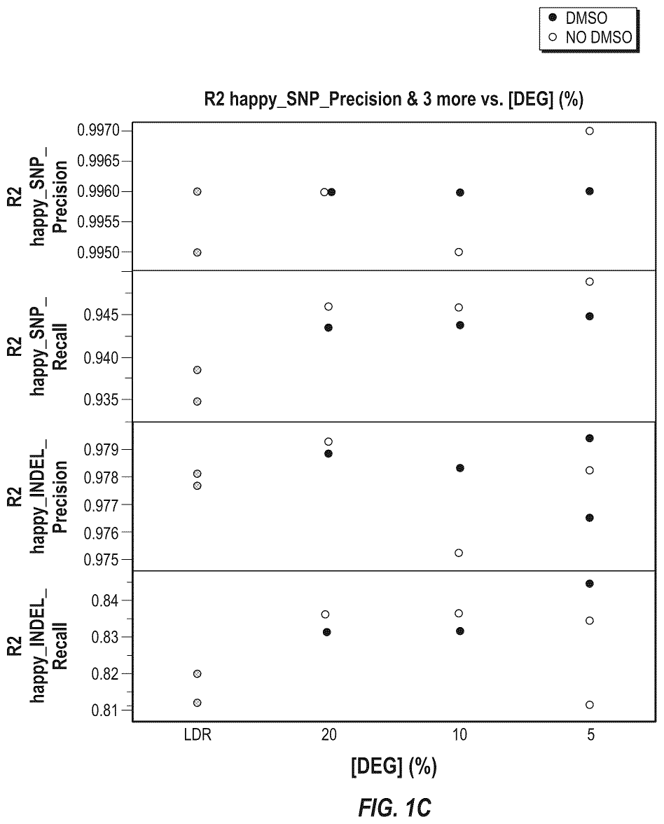

As shown in the data described below in Examples 2-6 and FIGS. 1A-5B, generally, the use of various DNA denaturant formulations that contain betaine resulted in identical sequencing yield compared to using formamide as the denaturant.

### Example 2—Comparable Metrics for DNA Denaturant Formulations Containing Betaine with and without DMSO Vs. Formamide

Human 450 bp libraries were sequenced as described in Example 1. DNA was denatured using different denaturant formulations containing betaine and using denaturants that utilized formamide, which functioned as a control. Sequencing metrics were performed on both Read 1 and Read 2 to determine the quality of the sequencing.

The results in FIGS. 1A-1C show that when DNA denaturant formulations are used without DMSO, DEG can be used to produce quality sequencing metrics.

The label “LDR” in each of FIGS. 1A-1C refers to a denaturant that utilized formamide (LDR). The labels “20”, “10”, and “5” in each of FIGS. 1A-1C refer to DNA denaturant formulations containing betaine, as described below.

Formulations with 20% DEG with DMSO and without DMSO (Label “20”)
Formulation with DMSO and 20% DEG:
50 mM CAPS pH 11
2.5M betaine
20% DEG
10% DMSO
   Formulation without DMSO and 20% DEG:
50 mM CAPS pH 11
2.5M betaine
20% DEG
   Formulations with 10% DEG with DMSO and without DMSO (label “10”)
   Formulation with DMSO and 10% DEG:
50 mM CAPS pH 11
2.5M betaine
10% DEG
10% DMSO
   Formulation without DMSO and 10% DEG:
50 mM CAPS pH 11
2.5M betaine
10% DEG
   Formulations with 5% DEG with DMSO and without DMSO (label “5”)
   Formulation with DMSO and 5% DEG:
50 mM CAPS pH 11
2.5M betaine
5% DEG
10% DMSO
   Formulation without DMSO and 5% DEG:
50 mM CAPS pH 11
2.5M betaine
5% DEG

### Example 3—Using Denaturing Formulations Containing Betaine in Bridge-Amplification

Denaturing formulations containing betaine were tested in bridge amplification. Human 450 bp libraries were sequenced as described in Example 1.

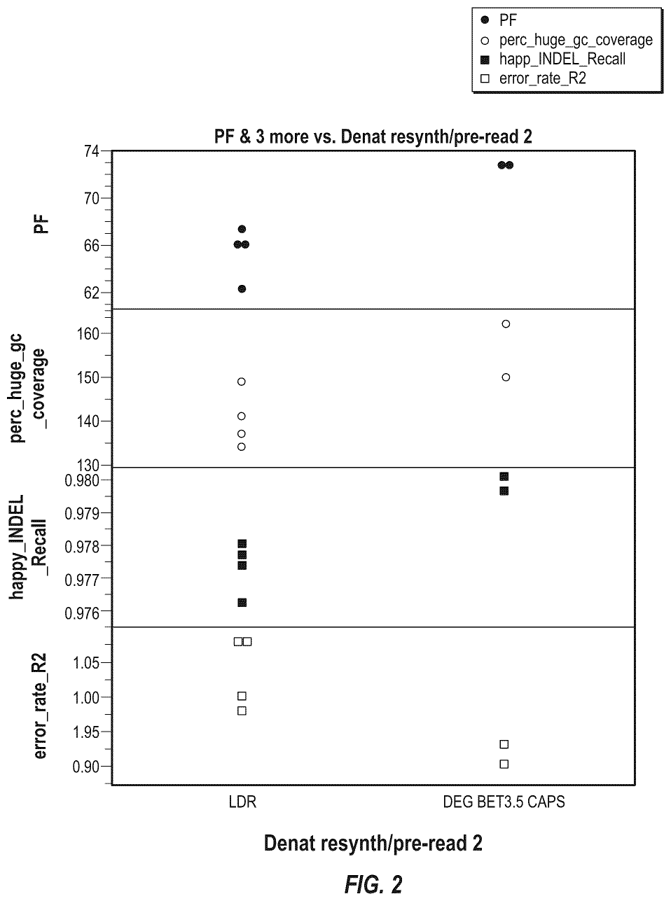

The label “LDR” in FIG. 2 refers to a denaturant that utilized formamide as the denaturant. The label DEG BET3.5 CAPS in FIG. 2 corresponds to the below formulation:
50 mM CAPS pH 11
3.5M betaine
20% DEG

As shown in the sequencing data in FIG. 2, relative to the LDR control, using the DEG BET3.5 CAPS denaturant resulted in a lower read 2 sequencing error rate. In addition, the DEG BET3.5 CAPS denaturant produced a pass filter percentage over 70%, which was higher than the pass filter percentage of the LDR control, which was less than 70%. This indicates that, unlike the control samples, using the BET3.5 CAPS denaturant resulted in highly pure cluster formation during bridge amplification.

### Example 4—Comparing Primary Sequencing Metrics Between Denaturants Containing Betaine and Denaturants that Utilize Formamide

Sequencing metrics were evaluated between denaturants containing betaine and denaturants that contain formamide. The ratios of library:denaturant:neutralizing buffer were adjusted and tested. Different formulations were tested as shown below:

### Ldr (Control)

99.9% Formamide

### DEG BET DMSO

50 mM CAPS pH 11
2.5M betaine
20% DEG
10% DMSO

### DEG30 BET

50 mM CAPS pH 11
2.5M betaine
30% DEG

### DEG BET3.5

50 mM CAPS pH 11
3.5M betaine
20% DEG

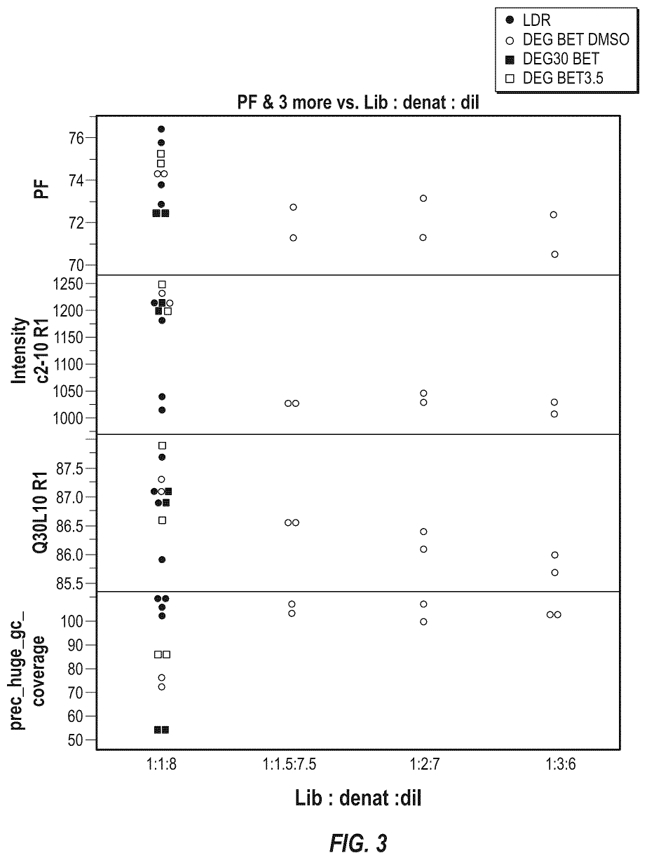

Each of the labels “LDR”, “DEG BET DMSO”, “DEG30 BET”, and “DEG BET 3.5” correspond to the labels on the x-axis on FIG. 3.

In FIG. 3, the ratio of 1:1:8 (library:denaturant:neutralizing buffer) was tested using each of the “LDR”, “DEG BET DMSO”, and “DEG30 BET” formulations. Only, the “DEG BET DMSO” formulation was used at the ratios of 1:1.5:7.5, 1:2:7, and 1:3:6.

As shown in FIG. 3, when using the DEG BEG DMSO, DEG30 BET, and DEG BET3.5 formulations, a ratio of 1:1.5:7.5 (library:denaturant:neutralizing buffer) was effective in producing good sequencing metrics. This is compared to LDR, which produced good sequencing metrics at a ratio of 1:1:8 (library:denaturant:neutralizing buffer).

### Example 5—Analyzing Sequencing Data Using DMSO-Free Versions of Denaturants Containing Betaine

Human 450 bp libraries were sequenced as described in Example 1. DMSO-free versions of the denaturants were tested. Different formulations were tested as shown below:

### LDR (Control)

99.9% Formamide

### DEG BET DMSO

50 mM CAPS pH 11
2.5M betaine
20% DEG
10% DMSO

### DEG30 BET

50 mM CAPS pH 11
2.5M betaine
30% DEG

### DEG BET3.5

50 mM CAPS pH 11
3.5M betaine
20% DEG

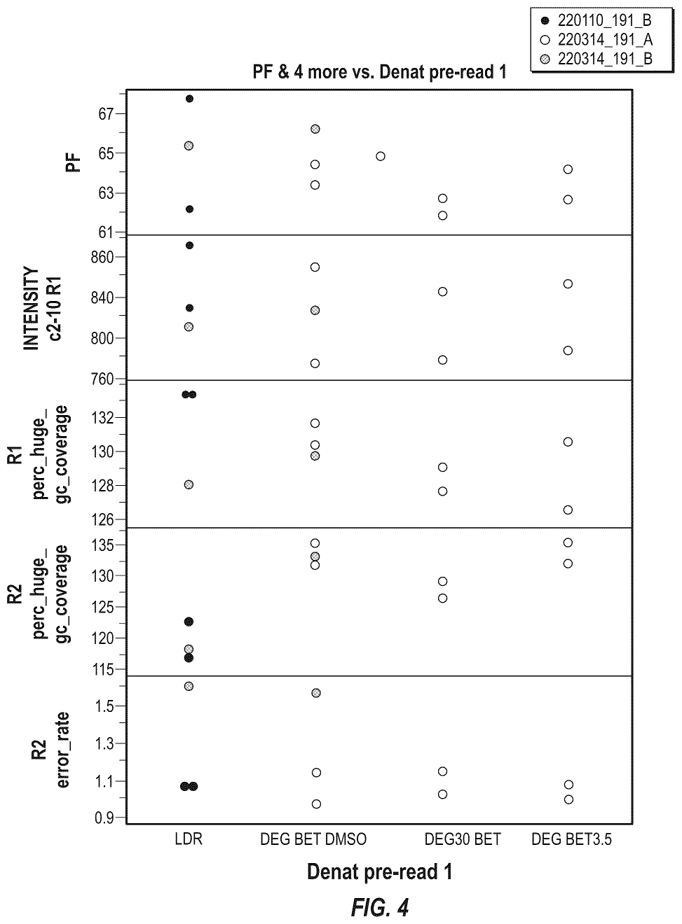

The “LDR”, “DEG BET DMSO”, “DEG30 BET”, and “DEG BET3.5” labels correspond to the labels on the x-axis of FIG. 4.

The sequencing data in FIG. 4 shows that the DNA denaturant formulations without DMSO (“DEG30 BET” and “DEG BET3.5”), showed an error rate and a pass filter rate similar to that of the DNA denaturant formulation with DMSO (“DEG BET DMSO”). This data indicates that DNA denaturant formulations without DMSO can be used in sequencing reactions when the concentration of betaine or DEG is increased.

### Example 6—Comparing Sequencing Performance Between a Denaturant Containing Betaine and a Denaturant that Contains Formamide, Over a 12-Month Period

Human 450 bp libraries were sequenced as described in Example 1. Sequencing metrics were tested for the reagents described below over a 12-month accelerated stability study at 60° C.

### LDR

99.9% Formamide

### DEG

50 mM CAPS pH11
2.5 M betaine
20% DEG
10% DMSO

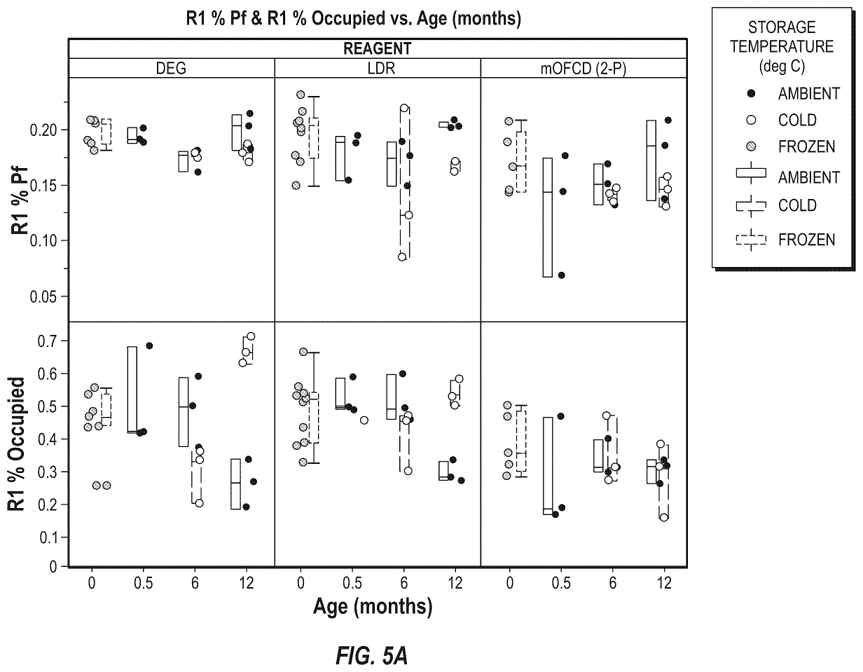

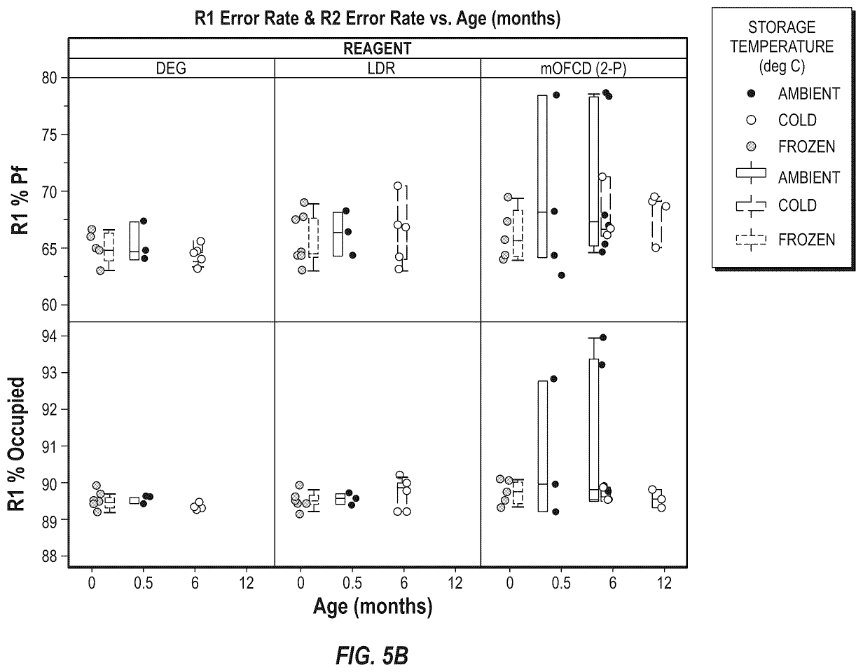

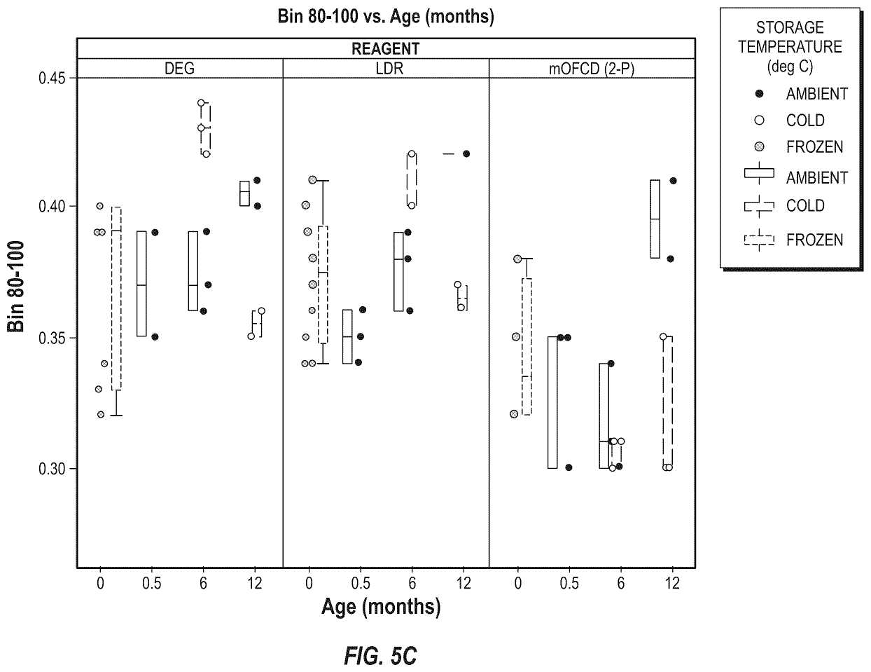

As shown in FIG. 5A, using the DEG formulation as a denaturant resulted in the pass filter rate of read 1 that was comparable to using the “LDR” formulation As shown in FIG. 5B, using the DEG formulation as a denaturant resulted in read 1 and read 2 error rates that were comparable to the error rates when using the LDR formulation. As shown in FIG. 5C, the DEG formulation resulted in comparable sequencing metrics to that of the LDR formulation.

### Example 7—Denaturing DNA Using Betaine and Gradual Addition of Other Components

In this example, the following denaturants were tested:

### Formamide

99.9% Formamide

### Betaine.

3.5M betaine

### Betaine+Buffer

50 mM CAPS pH11
3.5M betaine

### XDR-Betaine+Buffer+DEG

50 mM CAPS pH11
3.5M betaine
20% DEG

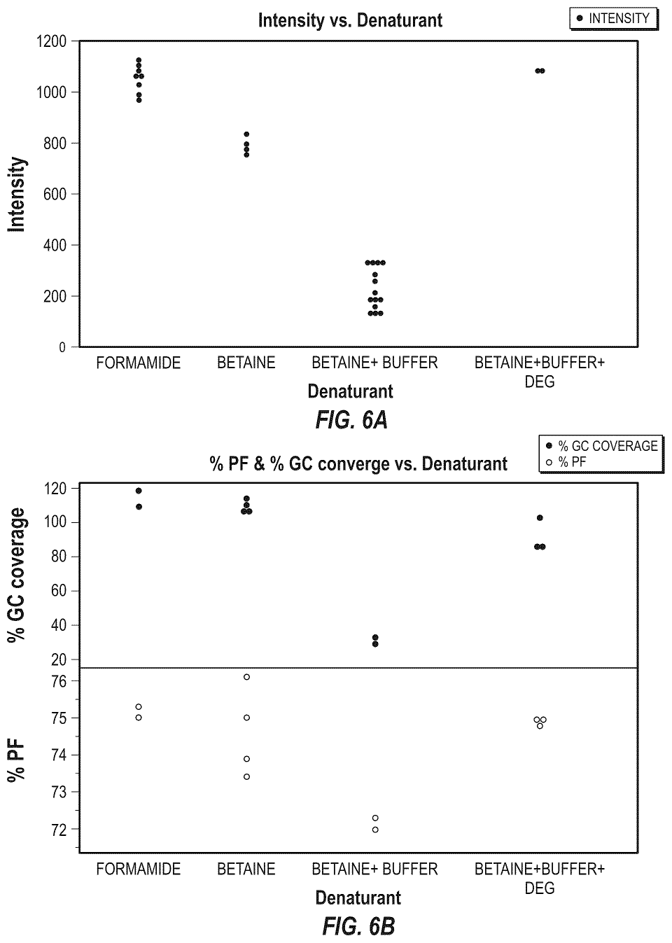

The labels “Formamide”, “Betaine,” “Betaine+Buffer”, and “Betaine+Buffer+DEG” correspond to the labels on the x-axes on both FIGS. 6A and 6B.

Denaturing after clustering: HiSeq X sequencing of human 450 bp libraries was performed using betaine as the denaturant along with other additives. 3.5 M betaine was used was used as a denaturant in place of formamide. The denaturant's ability to denature was measured with the ‘intensity’ metric (see FIG. 6A). If denaturing does not work, zero intensity will be measured using this metric.

The data shown in FIG. 6A shows successful denaturation of clusters when betaine was used as the denaturant. Adding buffer had a detrimental effect, which is shown in the “betaine+buffer” formulation. This detrimental effect is then mitigated by the addition of DEG, which is shown the “betaine+buffer+DEG” formulation. Temperature and contact time were identical across all the denaturants tested.

Denaturing libraries: HiSeq X sequencing of human 450 bp libraries was performed using betaine as the denaturant along with other additives. 3.5 M betaine was used as a denaturant in place of formamide. The ability of the denaturant to denature libraries was measured using percent GC (see FIG. 6B), which is very sensitive to incomplete denaturing. 3.5 M betaine with or without additives were used in place of formamide. The usual ratio of 1:1 (library to denaturant) was increased to 1:2 when denaturants other than formamide were used. Temperature and contact time were identical across all the denaturants tested.

As shown in FIG. 6B, the addition of buffer to denaturants containing betaine, even at high pH, reduces the ability of the formulation to denature DNA. This is evidenced by the reduction in percent GC coverage and the reduced pass filter percentage in the “betaine+buffer” formulation. Adding diethylene glycol recovers the ability of the reagent to denature DNA. This is evidenced by the increase in the percent GC coverage and the increase pass filter percentage in the “betaine+buffer+DEG” formulation relative to the “betaine+buffer” formulation. Thus, using the “betaine+buffer+DEG” formulation allows buffering of the reagent while still maintaining the ability of the reagent to denature DNA.

### Example 8—Range Finding of Betaine, DEG, and DMSO

In this example, the following denaturants were tested.

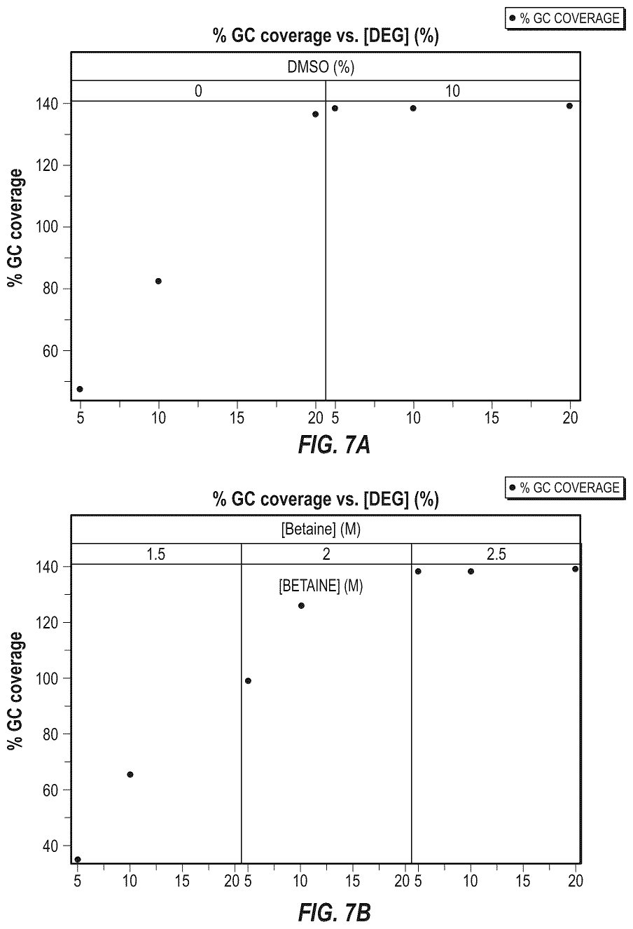

Denaturants tested in FIG. 7A:

Denaturants without DMSQ

### 5% DEG

50 mM CAPS pH11
2.5M betaine
5% DEG

### 10% DEG

50 mM CAPS pH11
2.5M betaine
10% DEG

### 20% DEG

50 mM CAPS pH11
2.5M betaine
20% DEG
   Denaturants with 10% DMSQ

### 5% DEG+10% DMSO

50 mM CAPS pH11
2.5M betaine
20% DEG
10% DMSO

### 10% DEG+10% DMSO

50 mM CAPS pH11
2.5M betaine
10% DEG
10% DMSO

### 20% DEG+10% DMSO

50 mM CAPS pH11
2.5M betaine
20% DEG
10% DMSO
   Denaturants tested in FIG. 7B:
   Denaturants with 1.5M Betaine

### 5% DEG+1.5 M Betaine

50 mM CAPS pH11
1.5M betaine
5% DEG
10% DMSO

### 10% DEG+1.5 M Betaine

50 mM CAPS pH11
1.5M betaine
10% DMSO

### 20% DEG+1.5 M Betaine

50 mM CAPS pH11
1.5M betaine
20% DEG
10% DMSO
   Denaturants with 2.0 M Betaine

### 5% DEG+2.0 M Betaine

50 mM CAPS pH11
2M betaine
5% DEG

### 10% DMSO10% DEG+2.0 M Betaine

50 mM CAPS pH11
2M betaine
10% DEG
10% DMSO

### 20% DEG+2.0 M Betaine

50 mM CAPS pH11
2M betaine
20% DEG
10% DMSO
   Denaturants with 2.5 M Betaine

### 5% DEG+2.5 M Betaine

50 mM CAPS pH11
2.5M betaine
5% DEG
10% DMSO

### 10% DEG+2.5 M Betaine

50 mM CAPS pH11
2.5M betaine
10% DEG
10% DMSO

### 20% DEG+2.5 M Betaine

50 mM CAPS pH11
2.5M betaine
20% DEG
10% DMSO

HiSeq X sequencing of human 450 bp libraries was performed using various XDR formulations as the denaturant. The DNA was denatured after clustering. Percent GC coverage was used as the metric, as this metric is sensitive to incomplete denaturation.

In FIG. 7A denaturant formulations with and without DMSO were tested. Data shows that denaturants that contain 10% DMSO and a range of 5-20% DEG worked well (see data points in FIG. 7A in which denaturant formulations were tested that contained 10% DMSO).

In FIG. 7B denaturant formulations with various concentrations of betaine and DEG were tested. Data shows that, in the presence of 2.5M betaine, a range of 5-20% DEG works well (see data points in FIG. 7B in which denaturant formulations were tested that contained 2.5 M betaine). When the concentration of betaine is 1.5M or 2.0M issues with GC coverage start to become apparent (see data points in FIG. 7B in which denaturant formulations were tested that contained 1.5M and 2.0M betaine).

### Example 9—Buffer Range Testing in XDR Formulations

In this example, the following denaturants were tested

Denaturants with 25 mM Buffer Molarity

### Buffer Molarity (25 mM) 10.0 pH

25 mM CAPS pH10
2.5M betaine
20% DEG
10% DMSO

### Buffer Molarity (25 mM) 11.0 pH

25 mM CAPS pH11
2.5M betaine
20% DEG
10% DMSO
   Denaturants with 50 mM Buffer Molarity

### Buffer Molarity (50 mM) 10.0 pH

50 mM CAPS pH10
2.5M betaine
20% DEG
10% DMSO

### Buffer Molarity (50 mM) 11.0 pH

50 mM CAPS pH11
2.5M betaine
20% DEG
10% DMSO

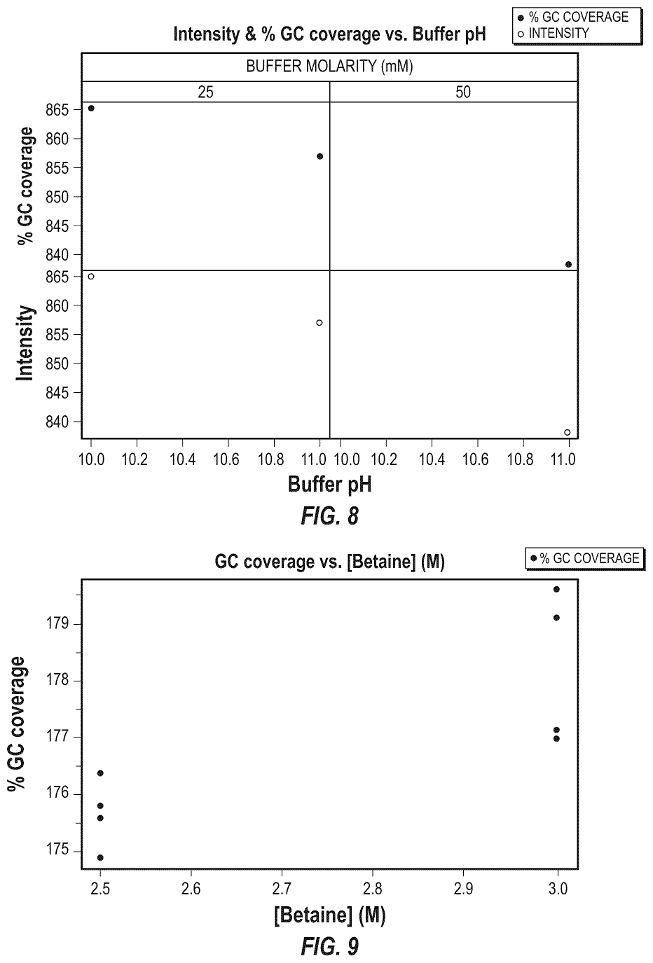

HiSeq X sequencing of human 450 bp libraries was performed using XDR formulations as the denaturant. The DNA was denatured after clustering. Percent GC coverage was used as one of the sequencing metrics, as this metric is the most sensitive to incomplete denaturing. In addition, sequence intensity was tested; when there is no denaturing the sequence intensity will be zero. The data in FIG. 8 shows that across the pHs that were tested, there was no difference in the quality of the sequencing between a buffer molarity of 25 and a buffer molarity of 50.

### Example 10—Testing Concentration Range of Betaine in XDR Formulations

In this example, the following denaturants were tested.

### Betaine (2.5 M)

50 mM CAPS
2.5 M betaine
20% DEG
10% DMSO

### Betaine (3.0 M)

50 mM CAPS
3.0 M betaine
20% DEG
10% DMSO

HiSeq X sequencing of human 450 bp libraries was performed using various XDR formulations as the denaturant. The DNA was denatured after clustering. Percent GC coverage was used as the metric, as this metric is sensitive to incomplete denaturing. The data in FIG. 9 shows that a denaturant formulation with 2.5 M betaine had a slight decrease in GC coverage relative to a denaturant formulation with 3.0 M betaine.

### Example 11—HybE Assay on XDR Conditions

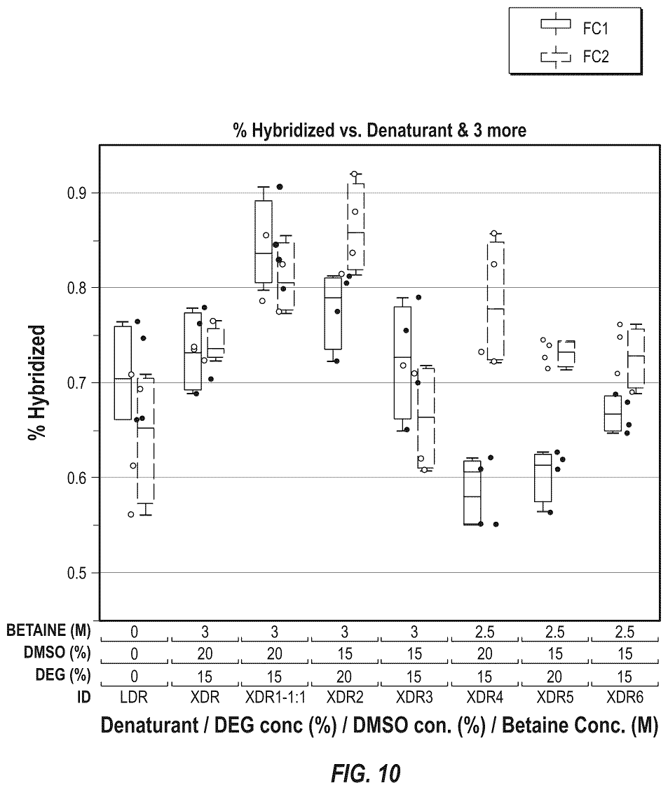

HybE is a surface-based assay that quantifies how much of a DNA library hybridized to the surface relative to the input concentration via a qPCR readout. In each of the tested formulations, a pH of 10.4 was used. As shown in FIG. 10, as measured by percent hybridization, betaine, DMSO, and DEG were each effective in denaturing DNA and they did not impact the subsequent efficiency of the library hybridization to the surface. The data in FIG. 10 suggests that the XDR formulations tested were as effective in denaturing DNA as the LDR control.

### Example 12—Exploring the Effects of DMSO Using a Mini-DOE

A mini high-powered DOE was generated using JMP software. The mini high-powered DOE was used to explore the effects of DMSO on library denaturation. The purpose of the assay was to determine whether formulations containing betaine that have lower concentrations of DMSO were still effective in denaturing DNA. Percent GC coverage was used as the metric, as this metric is sensitive to incomplete denaturing.

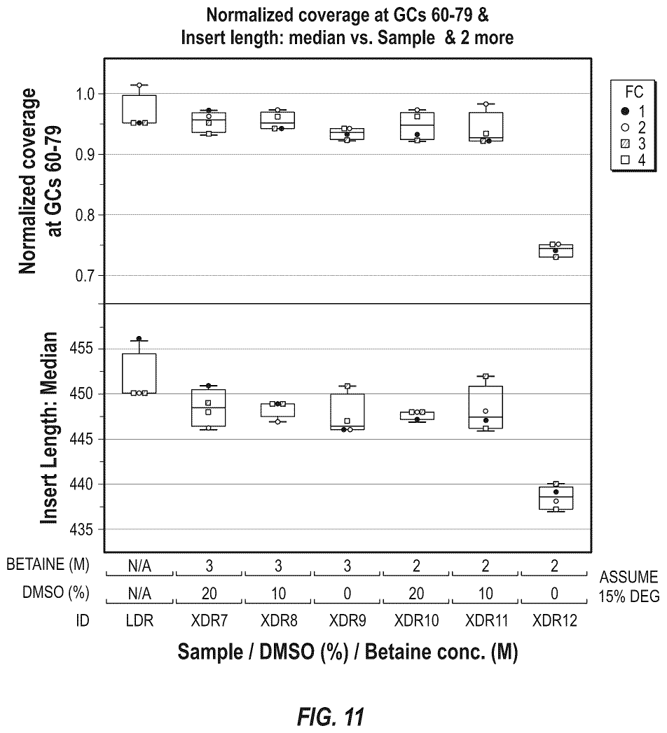

The data in FIG. 11 shows that the formulations containing betaine performed slightly worse in terms of GC coverage than the LDR formulation. Additionally, the data suggests that there is a slight bias toward shorter inserts. The data in FIG. 11 also show that each of the formulations containing betaine performed similarly, except for the formulation that contained no DMSO.

### Example 13—Testing XDR Formulations Using the NextSeg 2000

A 3-plex TruSeq DNA Nano human 450 bp DNA library was run on a NextSeq 2000 P3 kit 2×151+8+8. Analysis was performed using DRAGEN 3.9.5 Fluente orchestrated (N+3 shown are data from each index, only 1 run per condition).

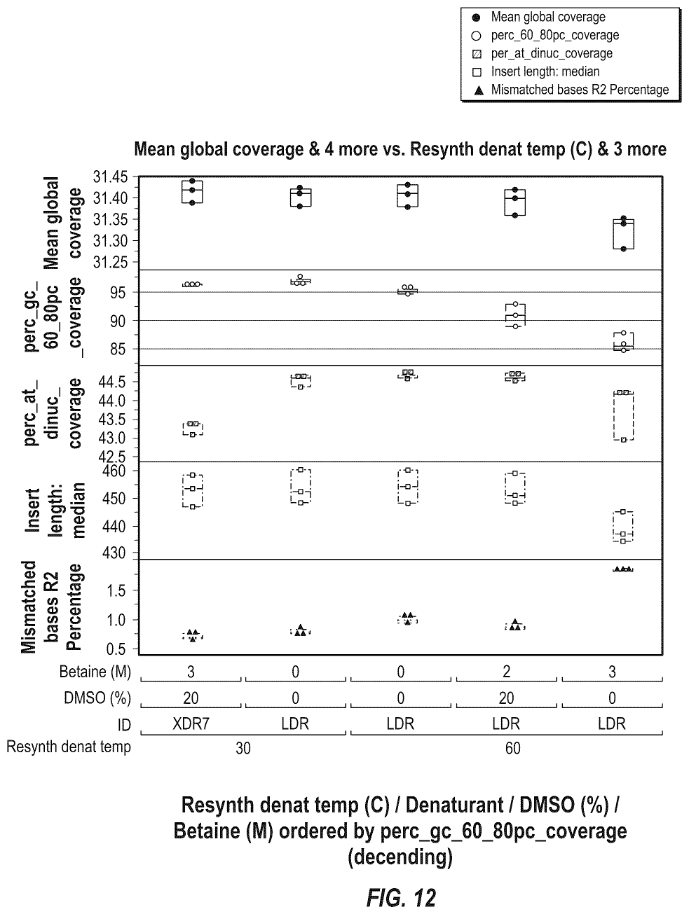

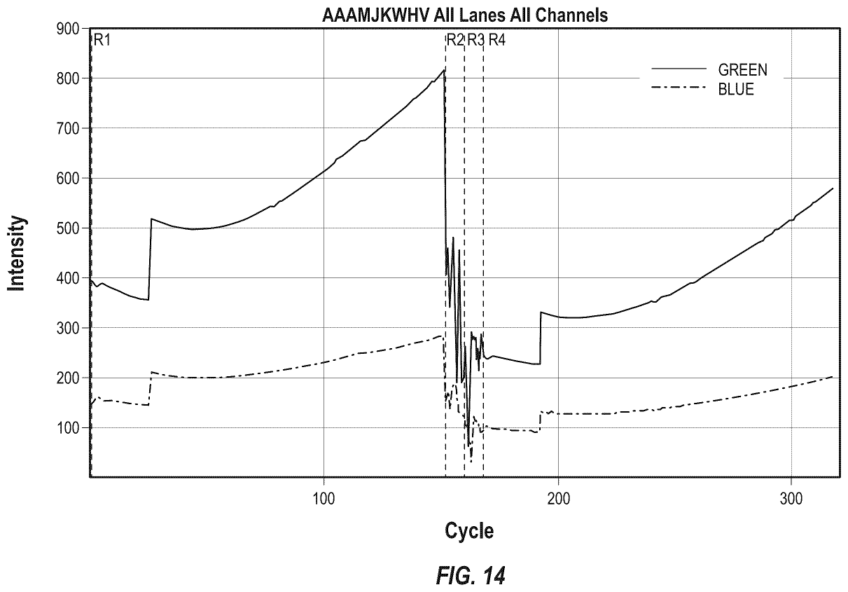

As shown in FIG. 12, low betaine or low DMSO concentrations in the denaturant formulations showed lower GC coverage compared to LDR formulations. Also, as shown in FIG. 12, high betaine and high DMSO in the denaturant formulations show similar performance to LDR; however, the denaturant formulations did show lower percent GC coverage relative to the LDR formulations. Further, as shown in FIG. 12, resynthesis at 60° C. showed no improvement with either the formulations containing betaine or LDR formulations. As shown in FIG. 14 and Table 1, the XDR14 formulation produced quality sequencing metrics.

TABLE 1

| | | Projected | | | | | % >= Q |
|---|---|---|---|---|---|---|---|
| | Yield | Total | Aligned | Error | Intensity | X >= | 30 (last |
| Level | Total (G) | Yield (G) | (%) | Rate (%) | Cycle 1 | Q30 | 10 cycles) |
| Read 1 | 205.73 | 205.73 | 0.00 | NaN | 148 | 92.34 | 87.71 |
| Read 2 (I) | 9.60 | 9.60 | 0.00 | NaN | 156 | 59.96 | 59.96 |
| Read 3 (I) | 9.59 | 9.59 | 0.00 | NaN | 123 | 79.05 | 79.05 |
| Read 4 | 205.64 | 205.64 | 0.00 | NaN | 92 | 77.15 | 68.79 |
| Non-Indexed Total | 411.37 | 411.37 | 0.00 | NaN | 120 | 84.75 | 78.25 |
| Total | 430.56 | 430.56 | 0.00 | NaN | 130 | 84.07 | 74.65 |

### Example 14—Using HiSeg X with XDR Formulations

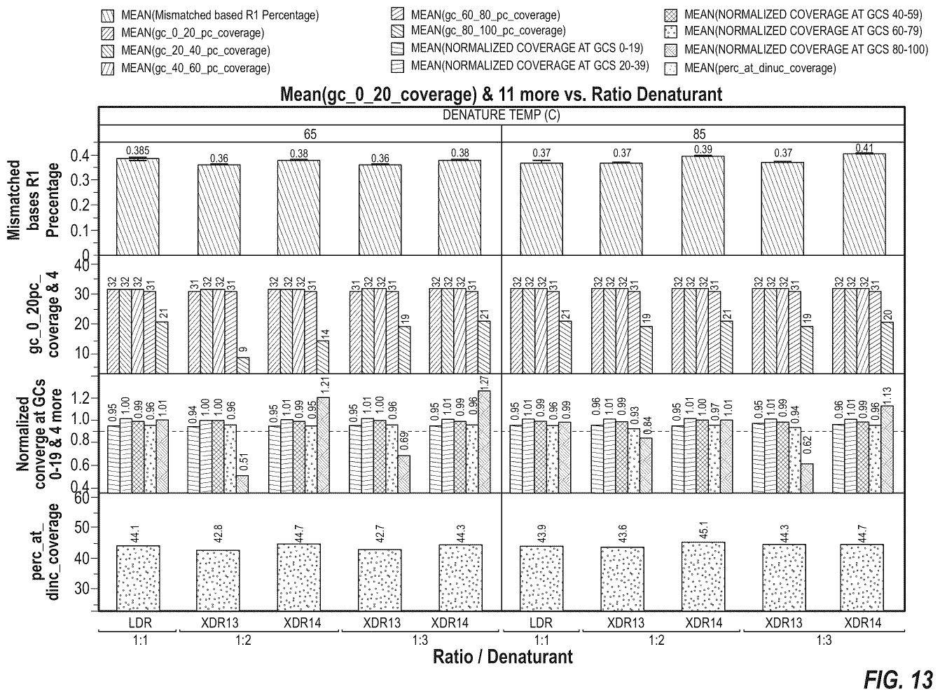

Various denaturing formulations containing betaine were tested using HiSeq X. FIG. 13 shows that increasing the concentration of DMSO (e.g., XDR14) can improve the GC percentage. In addition, FIG. 13 shows that increasing the XDR formulation to library ratio improves underperforming conditions. Further, FIG. 13 shows that increase the denaturing temperature to 85° C. provides more robust denaturing when compared to using a denaturing temperature of 65° C.

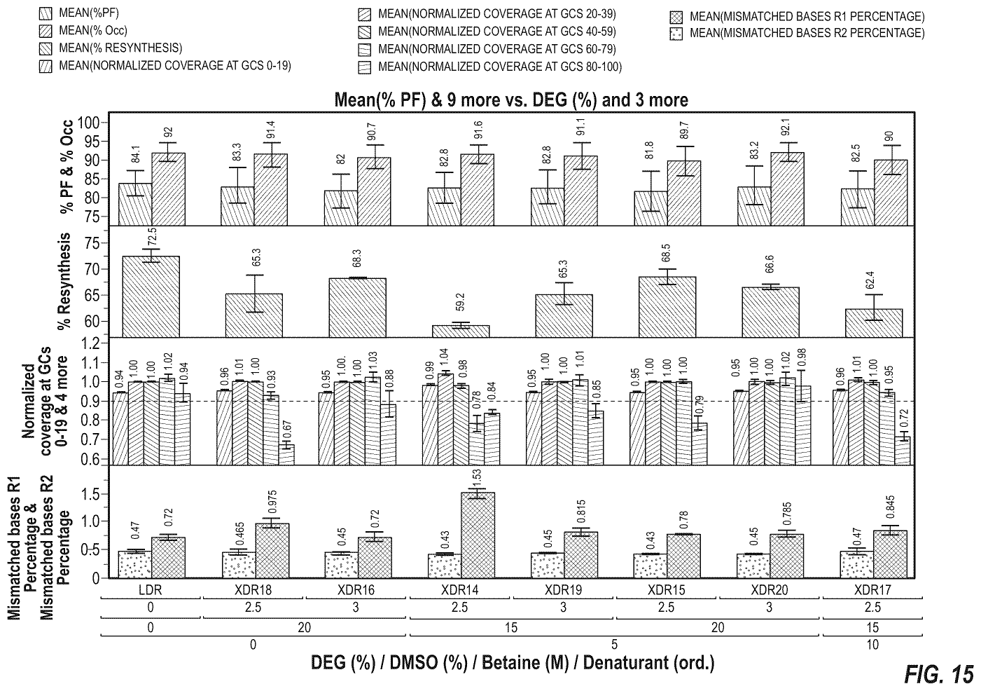

FIG. 15 shows that some of the XDR formulations give similar resynthesis percentage, PF and occupancy as the LDR control, as well as GC coverage and error rates. Further, FIG. 15 shows that XDR formulations that have betaine at a concentration of 3M improve sequencing metrics, relative to lower concentrations of betaine.

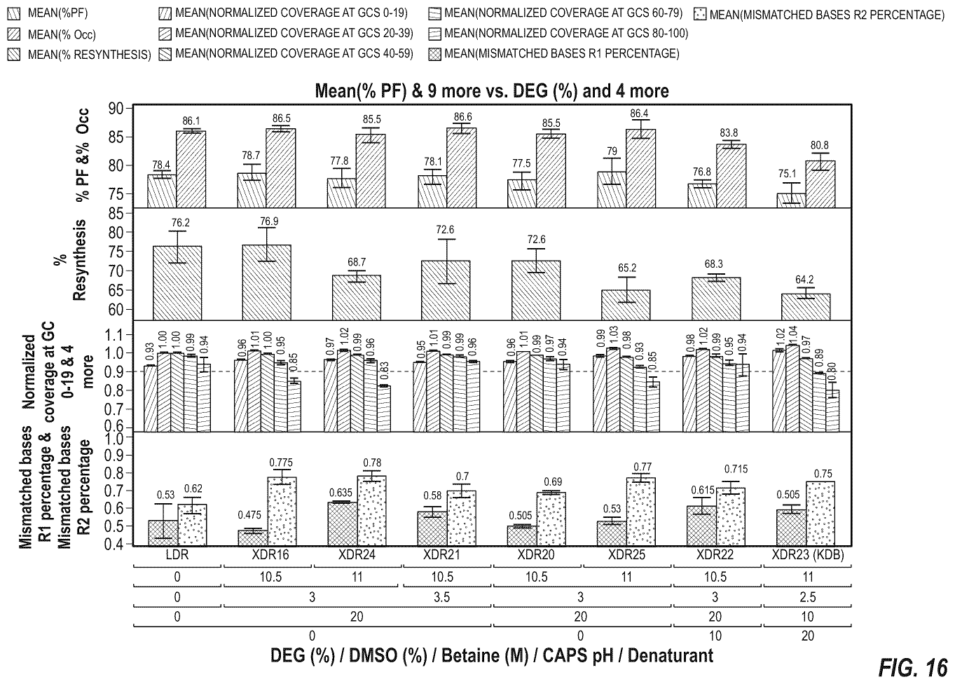

XDR formulations that contained no DEG were also tested using the HiSeq X. The purpose of testing XDR formulations without DEG was to mitigate high viscosities in certain XDR formulations. FIG. 16 shows that the XDR20 and XDR21 formulations produced robust sequencing metrics.

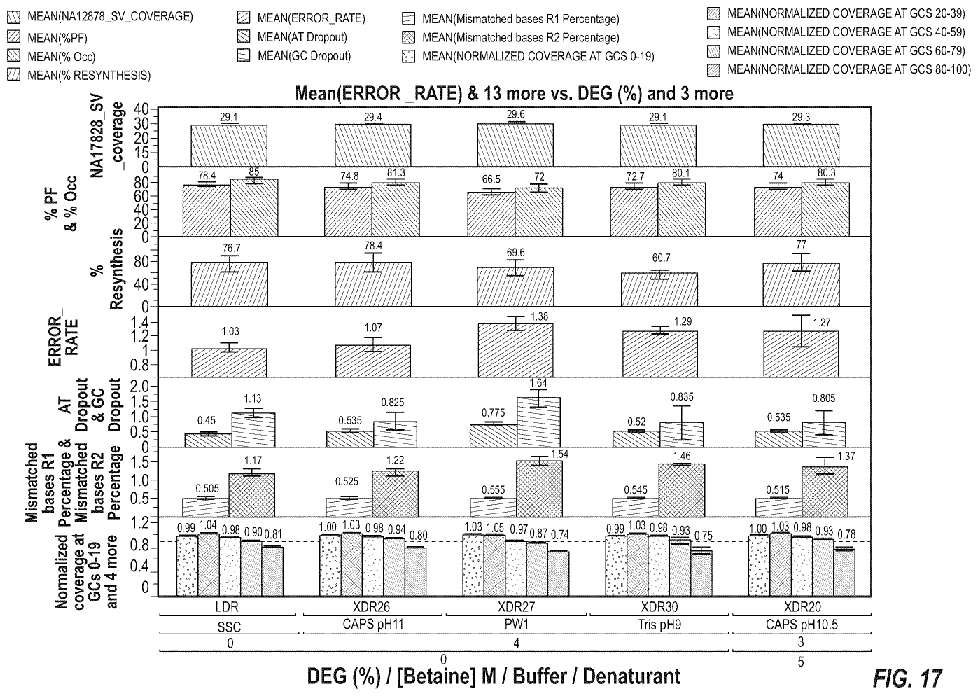

XDR formulations that contained different buffers were also tested using the HiSeq X. FIG. 17 shows that the XDR26 (an XDR formulation that contains CAPS pH 11 buffer) outperformed XDR27 and XDR30, which contain PW1 and Tris pH 9 buffers, respectively.

### Example 15—Testing for Viscosity in XDR Formulations

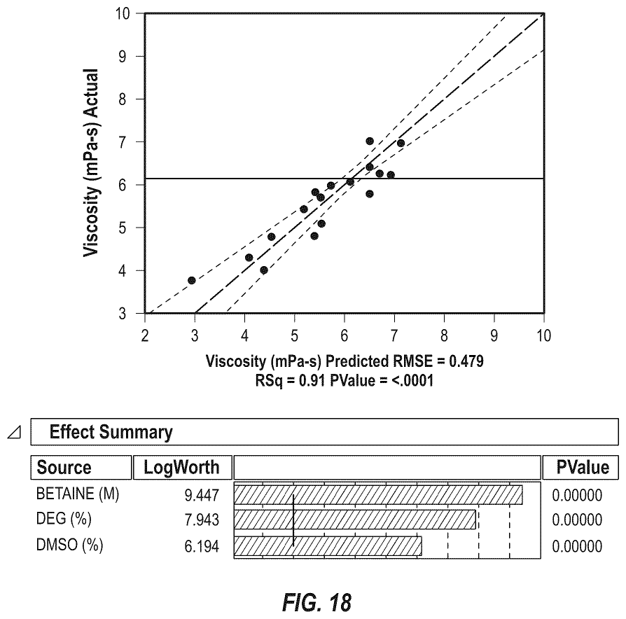

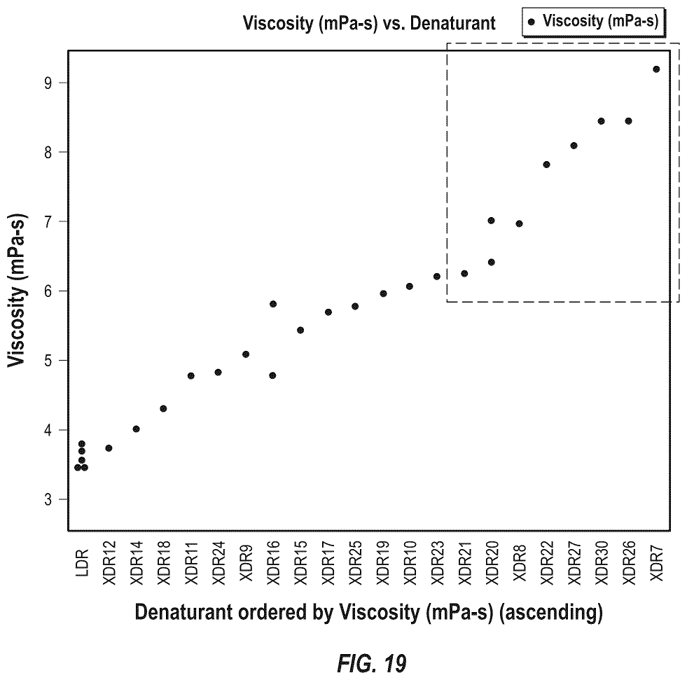

Using a viscometer, XDR formulations were tested to determine their viscosity. FIG. 18 shows that XDR formulations have a significantly higher viscosity than LDR. FIG. 19 shows that the XDR formulations with the highest viscosity generally correlated with the XDR formulations that performed the best in sequencing reactions.

### Additional Comments

While various illustrative examples are described above, it will be apparent to one skilled in the art that various changes and modifications may be made therein without departing from the disclosure. The appended claims are intended to cover all such changes and modifications that fall within the true spirit and scope of the examples provided herein.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A solution formulation for denaturing double-stranded DNA (dsDNA), the solution formulation comprising:
betaine; and
at least one organic solvent.

2. The solution formulation of claim 1, wherein the at least one organic solvent comprises diethylene glycol (DEG).

3. The solution formulation of claim 2, wherein a concentration of betaine in the solution formulation is between about 1M and about 5M, and a concentration of DEG in the solution formulation is between about 3% and about 40%.

4-6. (canceled)

7. The solution formulation of claim 1, wherein the concentration of betaine in the solution formulation is between about 2M and about 5M.

8-10. (canceled)

11. The solution formulation of claim 1, wherein the at least one organic solvent comprises diethylene glycol (DEG) and dimethyl sulfoxide (DMSO).

12. The solution formulation of claim 11, wherein a concentration of betaine in the solution formulation is between about 1M and about 5M, and a concentration of the DEG in the solution formulation is between about 3% and about 40%.

13. The solution formulation of claim 11, wherein a concentration of DMSO in the solution formulation is between about 5% and about 30%.

14-24. (canceled)

25. The solution formulation of claim 1, wherein the at least one organic solvent comprises DMSO.

26-30. (canceled)

31. The solution formulation of claim 25, wherein a concentration of DMSO in the solution formulation is between about 5% and about 30%.

32-35. (canceled)

36. The solution formulation of claim 1, further comprising a zwitterionic buffer.

37. The solution formulation of claim 36, wherein the zwitterionic buffer produces a pH in the solution formulation at a range of between about 9.5 and about 11.5.

38. The solution formulation of claim 36, wherein the zwitterionic buffer comprises any one or more of 4-(cyclohexylamino)-1-butanesulfonic acid (CABS), N-cyclohexyl-2-aminoethanesulfonic acid (CHES), 3-(cyclohexylamino)-2-hydroxy-1-propanesulfonic acid (CAPSO), N-cyclohexyl-3-aminopropanesulfonic acid (CAPS), and 3-((3-cholamidopropyl)dimethylammonio)-1-propanesulfonate) (CHAPS).

39. A method of denaturing double-stranded DNA (dsDNA), the method comprising:
contacting the dsDNA in a fluid-filled container with a denaturant comprising betaine.

40-45. (canceled)

46. The method of claim 39, wherein the denaturant further comprises diethylene glycol (DEG).

47. The method of claim 39, wherein the denaturant further comprises dimethyl sulfoxide (DMSO).

48. The method of claim 39, wherein the denaturant further comprises a zwitterionic buffer.

49. The method of claim 48, wherein the zwitterionic buffer produces a pH in the fluid-filled container between about 9.5 and about 11.5.

50. A kit for denaturing double-stranded DNA (dsDNA), the kit comprising:
a denaturant comprising betaine and diethylene glycol (DEG) in a solution; and
a buffer.

51. The kit of claim 50, wherein a concentration of DEG in the solution is between about 20% and about 40%.

52. The kit of claim 50, wherein the denaturant further comprises dimethyl sulfoxide (DMSO).

53. (canceled)

54. The kit of claim 50, wherein a concentration of betaine in the solution is between about 1M and 5M.

55-59. (canceled)

60. The method of claim 39, wherein a ratio of denaturant to the dsDNA is between about 1:1 and about 3:1.
